(19) United States
(12) Patent Application Publication
(10) Pub. No.: US 2023/0329185 A1
(43) Pub. Date: Oct. 19, 2023

(54) MILKING APPARATUS OPERATIONAL TOLERANCE MONITORING SYSTEM

(71) Applicant: Nathan Lax, Brandon, SD (US)

(72) Inventor: Nathan Lax, Brandon, SD (US)

(21) Appl. No.: 17/720,560

(22) Filed: Apr. 14, 2022

(51) Int. Cl. A01J 5/007; A61L 2/18

(52) U.S. Cl. CPC A01J 5/007; A61L 2/18; A61L 2202/15; A61L 2202/14

(57) ABSTRACT

A milking apparatus operational tolerance monitoring system includes a milk collection apparatus for milking cattle including a plurality of components. A monitoring and warning system is in communication with the milk collection apparatus to detect proper functioning of the components. At least some of the components include a detector capable of reading a measurement that is characteristic of that component. An interface is in communication with a processor and the detectors to provide an alert when one of the measurements is outside of a standard parameter for that measurement. The alert is an audible and/or visual alert and indicates which component is associated with the measurement that is outside of its standard parameter.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to systems monitoring device and more particularly pertains to a new systems monitoring device for milking assemblies to ensure proper functionality of the milking assemblies as well as warning systems for alerting personnel that the milk collection apparatus is not properly functioning.

### (2) Description of Related Art Including Information Disclosed Under 37 CFR 1.97 and 1.98

The prior art relates to systems monitoring devices that are often used with milking assemblies to alert persons that the milk collection apparatus is not functioning as intended. Prior warning systems have been made for milking assemblies; however, their usage has been limited to a overall, generic types of malfunction warnings. That is, though a milk collection apparatus includes a large number of interacting mechanisms, the prior warning systems have only indicated a problem with the milk collection apparatus as a whole and have not addressed the individual mechanism which has failed. An operator of such an assembly would consequently know that a failure has occurred but would not know the point of failure leading to the time-consuming task of locating the problem. Moreover, some potential failures such as vacuum suction may require a complete system shut down while those including refrigeration are potentially correctable while milking is ongoing. Thus, there is a need for an alert system that more quickly informs a user of a milk collection apparatus as to the precise reason an alert has been initiated.

### (h) BRIEF SUMMARY OF THE INVENTION

An embodiment of the disclosure meets the needs presented above by generally comprises a milk collection apparatus for milking cattle. The milk collection apparatus includes a plurality of components in fluid and/or thermal communication with each other for milking a cow and to transport collected milk for storage. A monitoring and warning system is in communication with the milk collection apparatus to detect proper functioning of the components of the milk collection apparatus. The monitoring and warning system indicate operation of multiple ones of the components independently of other ones of the components. The monitoring and warning system includes a processor and a plurality of detectors. At least some of the components include a detector capable of reading a measurement that is characteristic of that component. The detectors send the measurements of the components to the processor and the processor is programmed to determine if any of the measurements are outside of a standard parameter. An interface is in communication with the processor and provides an alert when one of the measurements is outside of a standard parameter for that measurement. The alert is an audible and/or visual alert and indicates which component is associated with the measurement that is outside of its standard parameter.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a schematic view of a pipeline milking assembly of a milking apparatus operational tolerance monitoring system according to an embodiment of the disclosure.

FIG. 2 is a schematic of a cleaning solution assembly view of an embodiment of the disclosure.

FIG. 3 is a schematic view of a cooling assembly of an embodiment of the disclosure.

FIG. 4 is a schematic electrical block diagram view of an embodiment of the disclosure.

FIG. 5 is a schematic block diagram view of an embodiment of the disclosure.

FIG. 6 is a schematic block diagram view of an embodiment of the disclosure depicting one communication route between the system described herein and personal computing devices.

FIG. 7 is a schematic block view of an embodiment of the disclosure depicting numerous detectors of one embodiment.

FIG. 8 is a schematic of a bulk tank cooling assembly of an embodiment of the disclosure.

### (j) DETAILED DESCRIPTION OF THE INVENTION

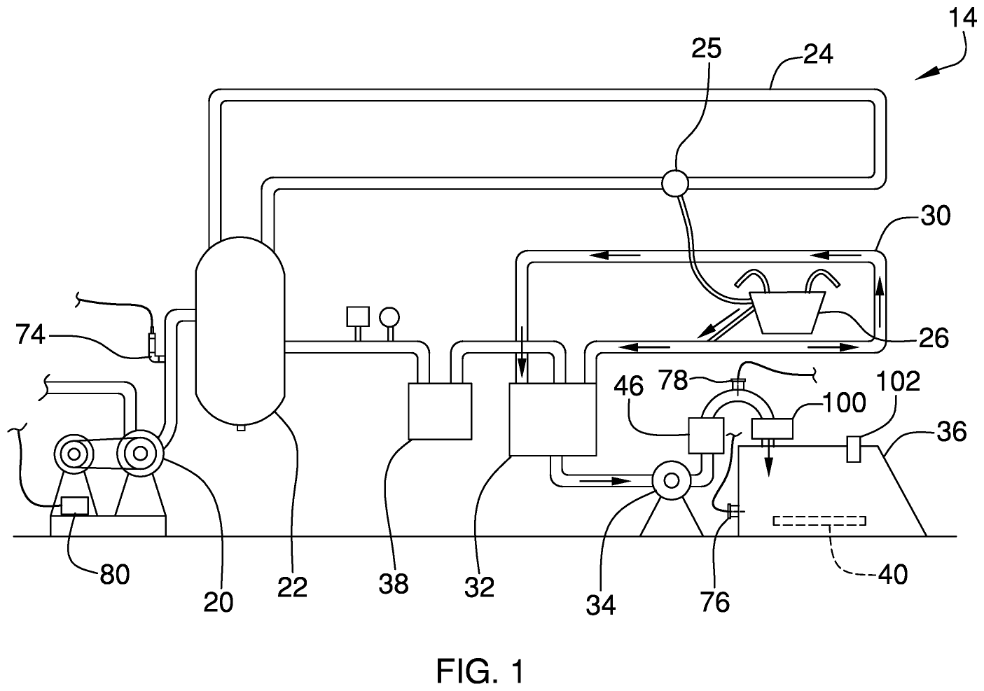

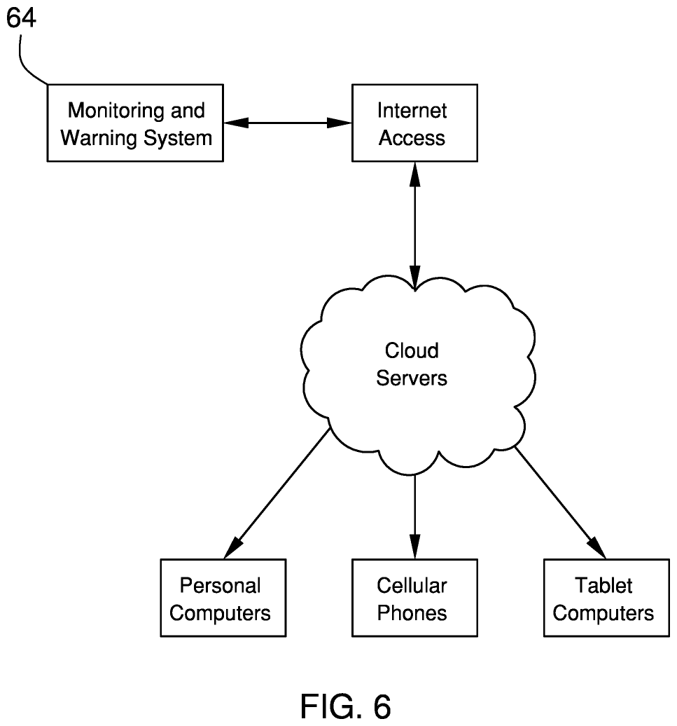

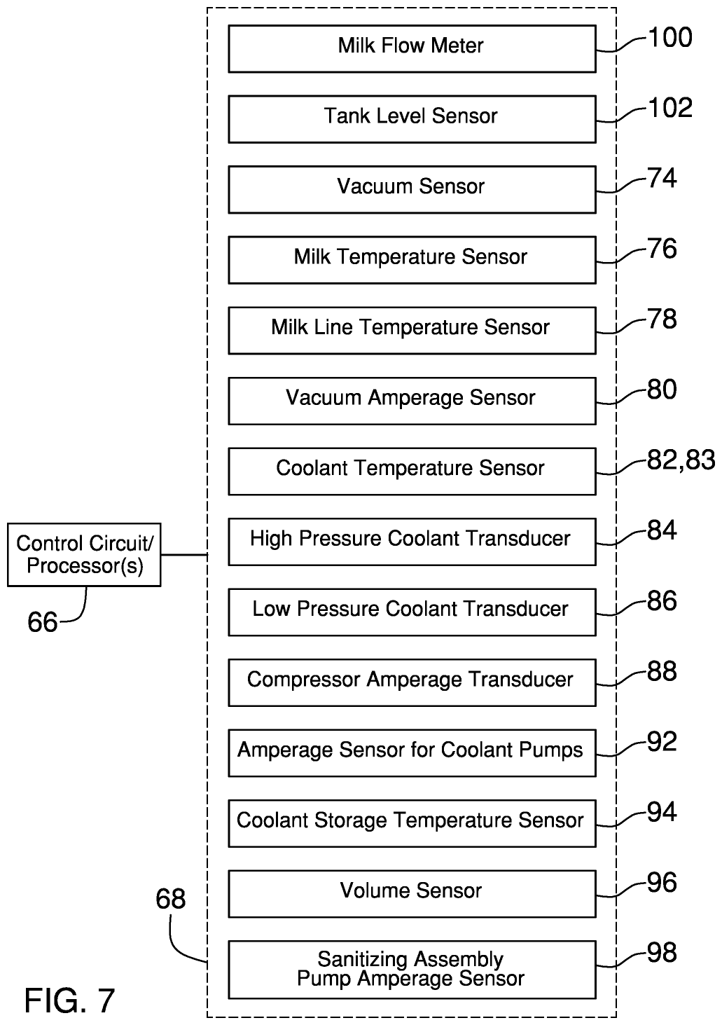

With reference now to the drawings, and in particular to FIGS. 1 through 8 thereof, a new systems monitoring device embodying the principles and concepts of an embodiment of the disclosure and generally designated by the reference numeral 10 will be described.

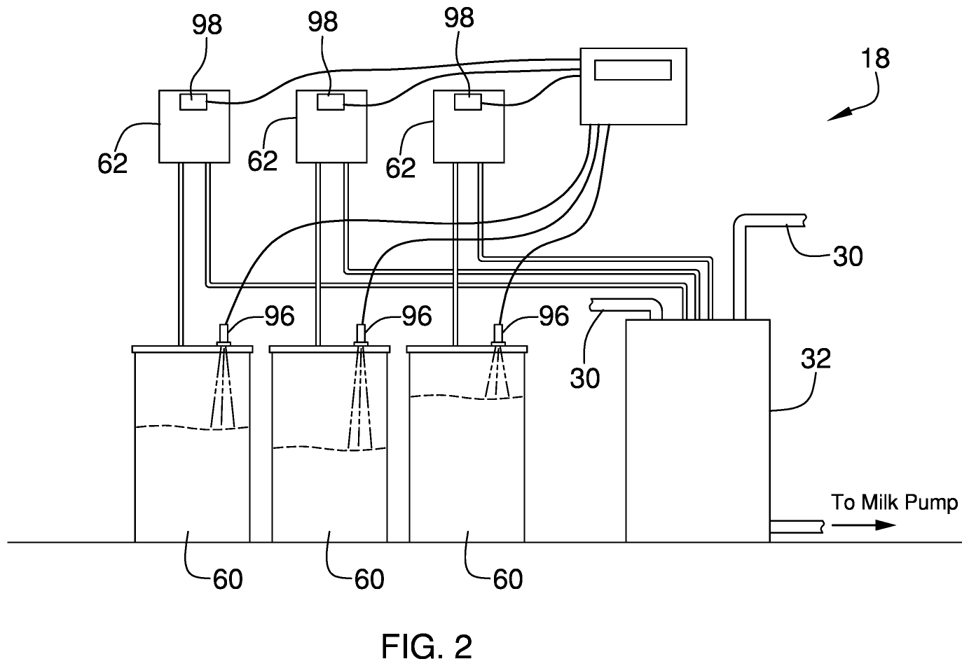

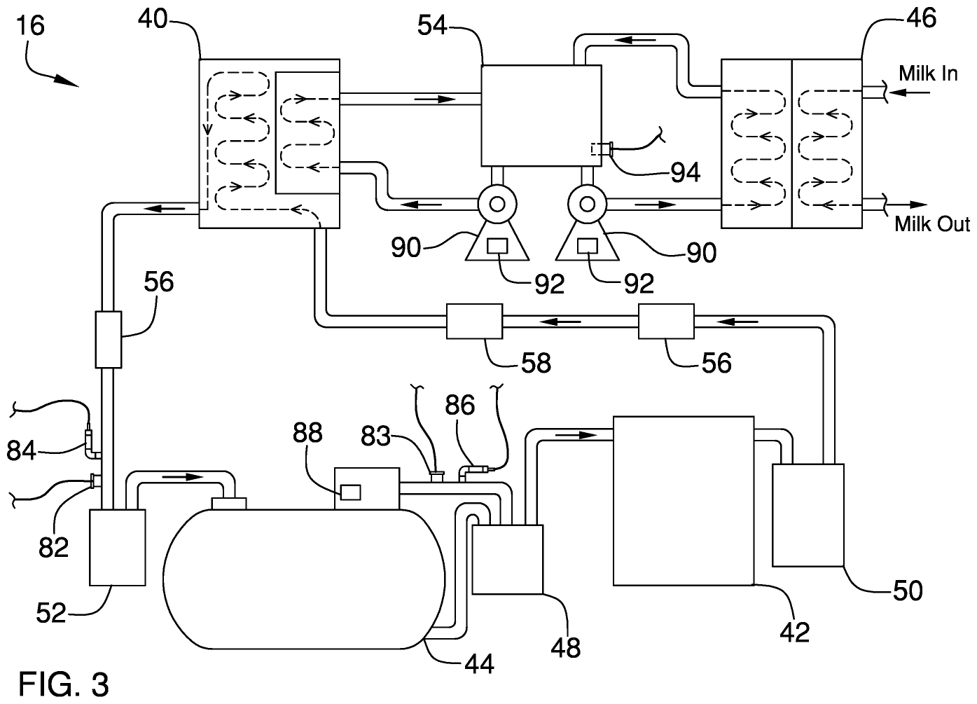

As best illustrated in FIGS. 1 through 8, the milking apparatus operational tolerance monitoring system 10 generally comprises a milk collection apparatus 12 comprising a milking assembly 14 shown in FIG. 1, a cooling assembly 16 shown in FIG. 3, and a sanitizing assembly 18 shown in FIG. 2 wherein components thereof are in thermal or fluid communication with each other. It should be understood that FIGS. 1-3 are only one embodiment of schematic versions of the various assemblies and each may be configured as needed or modified for their particular location and usage.

The milking assembly 14 collects milk directly from cows and includes a plurality of conventional components in fluid communication with each other for milking a cow and to transport the milk for storage. The plurality of components typically includes a vacuum pump 20 fluidly coupled to a reserve tank 22. A pulsator line 24 is fluidly coupled to the reserve tank 22 and a milking unit 26 is fluidly coupled to the pulsator line 24. Generally, a milking assembly 14 will include multiple milking units 26 each comprising multiple cups which are manipulated by the pulsator line 24 changes in pressure to draw milk from teats of the cow. A milk line 30 is fluidly coupled to the milking unit 26 and to the reserve tank 22. A receiver jar 32 fluidly, coupled to the milk line 30 between the milking unit 26 and the reserve tank 22, captures the milk while air flow from the milk line 30 continues to the reserve tank 22. A milk pump 34 fluidly coupled to the receiver jar 32 carries fluid, i.e. milk, to a milk tank 36 fluidly coupled to the milk pump 34. Specifically, milk received by the milking unit 26 flows into the receiver jar 32 from the milk line 30 and thereafter the milk pump 34 pumps the milk from the receiver jar 32 into the milk tank 36. The milk tank 36 may include a free standing tank or milk storage and transportation vessels such as are utilized with semi-tanks and pulled by a semi-trucks. Additional components include a sanitary milk trap 38 that is in fluid communication with the milk line 30 between the receiver jar 32 and the reserve tank 22 to prevent milk from entering the reserve tank 22.

In most conventional milking systems, the cooling assembly 16, colloquially known as a “chiller”, is utilized for reducing the temperature of the milk and ensuring the milk is held at a sufficiently cooled temperature. The cooling assembly 16 includes an evaporator 40, a condenser 42, and a compressor 44 being in fluid communication with each other. The cooling assembly 16 may include a plate cooler 46 through which the milk line 30 flows to lower the temperature of the milk as it travels from the milk pump 34 to the milk tank 36. Alternatively, or in addition to the plate cooler 46, the evaporator 40 may be mounted on the milk tank 36 to cool the milk within the milk tank 36. In one embodiment, as can be seen in FIG. 3, the compressor 44 is fluidly coupled to, in turn, a separator 48, condenser 42, receiver 50, evaporator 40, and accumulator 52. The evaporator 40 is in thermal communication with lines supplying a coolant tank 54, which may be holding a glycol solution, that in turn is fluidly coupled to the plate cooler 46. Filters 56 and expansion valves 58 may be utilized as is necessary and customary in such cooling assemblies.

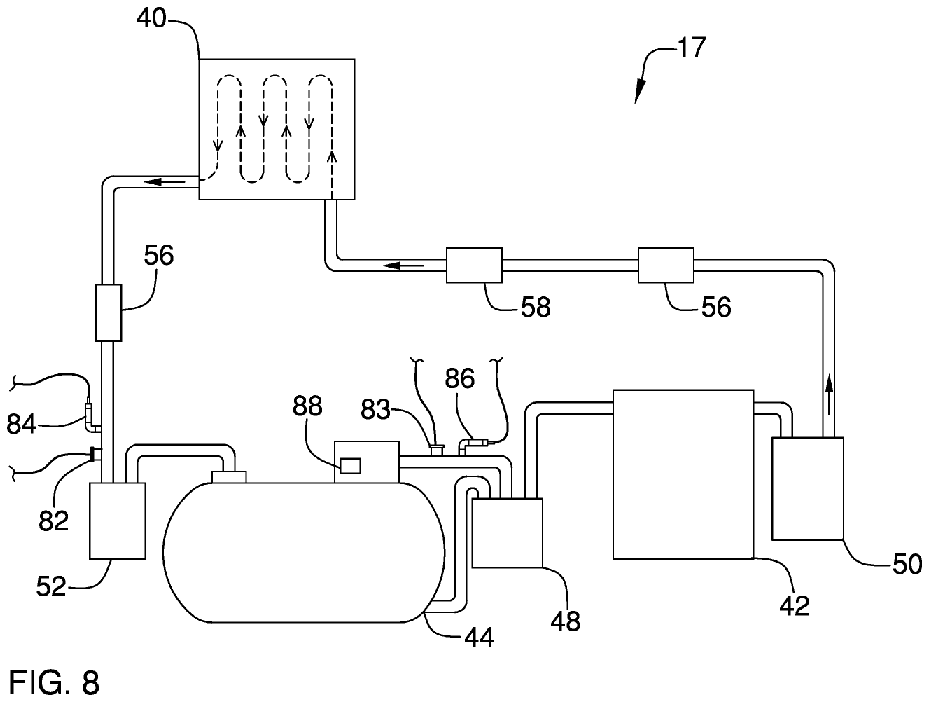

While the cooling assembly 16 as depicted in FIG. 3 may include the evaporator 40 being positioned within the tank 16, typically a secondary cooling assembly 17 as shown in FIG. 8 will be utilized. This secondary cooling assembly 17 includes the general structure of the cooling assembly 16 of FIG. 3, but would not include the coolant tank 54, such as for holding glycol, or the plate cooler 46. It should be understood that all monitoring systems and sensors described below that are used with the cooling assembly 16 would be duplicated for the secondary cooling assembly 17 as needed where the same structure is being utilized.

A milk collection apparatus 12 also generally includes the sanitizing assembly 18 which is in fluid communication with the milk line 30. The sanitizing assembly 18 includes a plurality of hold tanks 60 each configured to contain one of a plurality of cleaning agents. The cleaning agents may include any conventional cleaning agents utilized in the milking industry including detergents, alkaline additives, and sanitizing chemicals such as chlorine-based sanitizers. A plurality of cleaning pumps 62 is provided. Each of the cleaning pumps 62 is fluidly coupled to at least one of the hold tanks 60 and each hold tank 60 may include its own cleaning pump 62. Each of the cleaning pumps 62 is fluidly coupled or in fluid communication with the milk line 30 for pumping cleaning agents into the milk line 30 such that all areas where milk has travelled through the milking assembly 14 are cleaned. The multiple cleaning pumps 62 allows the user to determine how much of each cleaning agent is to be utilized.

A monitoring and warning system 64 is in communication with the milk collection apparatus 12 to detect proper functioning of the components of the milk collection apparatus 12. The monitoring and warning system 64 indicates the operation of each of the components of the milk collection apparatus 12 independently of other ones of the components. The monitoring and warning system 64 includes a processor 66 and a plurality of detectors 68. The processor 66 may include a plurality of plurality of processors or control units configured in a typical manner and which will further include conventional memory modules as needed. The monitoring and warning system 64 may be powered by the electrical power supply of the milk collection apparatus 12 or one of the components thereof, or may have its own dedicated power source which may further include a backup battery power supply.

At least some of the components of the milk collection apparatus 12 have operationally coupled thereto one of the detectors 68 capable of reading a measurement that is characteristic of that component. Thus, if a component is utilized to alter the air pressure within the milk collection apparatus 12, its associated detector 68 will measure air pressure. If the component alters the temperature of a component of the milk collection apparatus 12, its associated detector will measure temperature. The characteristic of the component is typically a parameter within which one can measure and determine if a recorded measurement is within known safe and correct operating standards. Since the components can vary depending on size, make, and electrical configuration, the parameters will often be determined as each component is incorporated into the overall system 10. The detectors 68 send the measurements of their associated components to the processor 66 and the processor 66 is programmed to determine if any of the measurements are outside of a standardized parameter. The monitoring and warning system 64 will typically be programmed to store the recorded measurements for later accessing as needed.

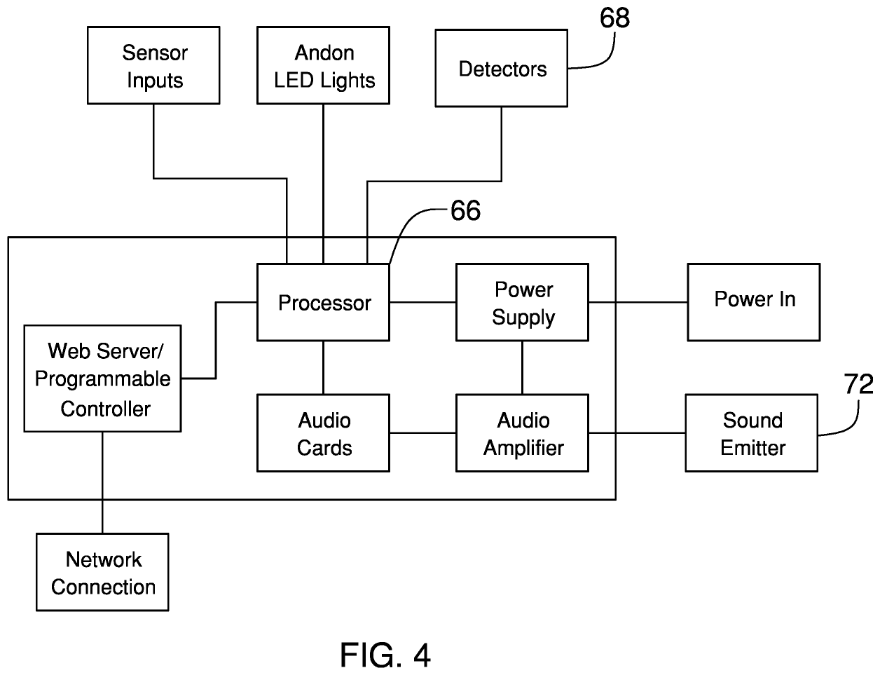

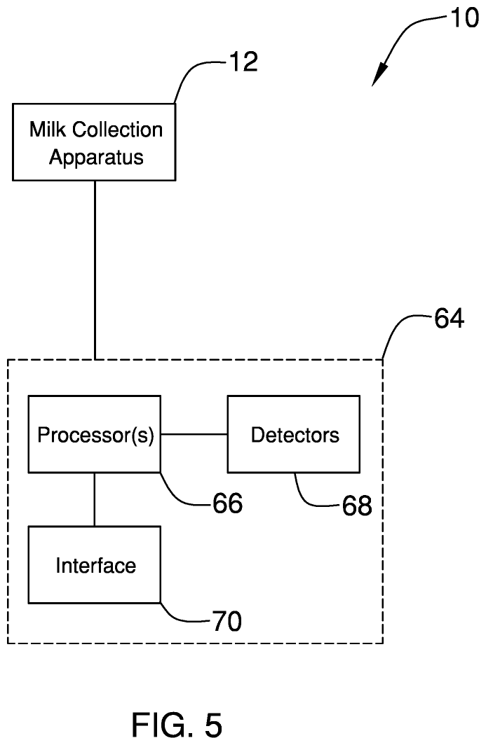

The monitoring and warning system 64 additionally includes an interface 70 to allow a user of the system 10 to receive information regarding the received measurements. The interface 70 is in communication with the processor 66 and provides an alert when one of the measurements is outside of the standard parameter for that measurement. Thus, if amperages, temperatures, pressures, etc., are not within the pre-determined allowed parameters, the alert will signal the user of the system 10, or others having access to the interface 70, that the milk collection apparatus 12 is not functioning properly. The alert will be an audible and/or visual alert, which may be localized and/or sent remotely. Moreover, the alert indicates the component associated with the measurement found to be outside of its standard parameter. The interface 70 will typically be programmed to provide a message report associated with any audible or visual alerts. The message report may be accessed any number of conventional ways which are more fully explained below. Furthermore, the message report, in addition to specifically identifying the source of the alert, may include pre-programmed suggestions as to the possible causes of alert as well as potential solutions for each possible cause. FIGS. 4 and 5 are for explanation purposes only and it should be understood that each of the milk collection 14, cooling 16 and sanitizing 18 assemblies or components thereof may include separate processors, detectors, memory modules and the like to accurately measure required parameters and these Figures are therefore not limiting as to circuit requirements.

The interface 70 will typically include any conventional electronic device capable of providing information regarding the system 10 to a user thereof. The interface may include a display, such as for example an electronic monitor, that is electrically coupled to the processor 66 to display the measurements. The display may be positioned directly on the milk collection apparatus 12, adjacently thereto, or remotely such as in an adjoining structure. The interface 70 may comprise a touch screen for allowing user input to also control various components as well as to bring up data concerning the measurements. However, the display may also be a conventional computer monitor connected to a computer and standard keyboard wherein the computer includes the processor 66 or is in communication with the processor 66 either by wired or wireless connection. Thus, a wireless transmitter may also be electrically coupled to the processor 66 to wirelessly transmit the measurements to a receiver for remote viewing of the measurements. This may be accomplished in any conventional manner. The processor 66 may be operationally coupled to a transmitter or wireless router, or a Bluetooth or WiFi transmitter, for example, may be within a same housing as the processor 66 to communicator with a wireless router. Additionally or alternatively, the transmitter may have cellular capabilities to access cellular phone networks.

It is also contemplated that the system 10 may be in communication with computers and/or cellular phones via a network connection to the internet utilizing hardlines or cellular signals. Cellular phones, tablets, computers, and other personal computing devices may include program applications for interpreting data from the measurements, controlling the system 10, and to actively receive the alerts utilizing well known protocols. Alternatively, or in addition to the program application, the personal computing devices may be used to access portals to cloud based servers containing the data from the system 10 and placed there by the network connection. However, it should be understood that the monitoring and warning system 10 may also send alerts directly to cellular phones, computers and the like without utilizing an internet-based program.

In addition to the above, the interface may further include a dedicated sound emitter 72 that is in communication with the processor 66 wherein the sound emitter 72 is mounted on, or adjacent to, the milk collection apparatus 12 such that a technician or other person in the immediate vicinity of the milk collection apparatus 12 will be able to quickly attend to the system 10. The sound emitter 72 emits one of a plurality of sounds when a measurement is outside of the standard parameter for that measurement. Additionally, each of the sounds may be different with respect to each other and is associated with only one of the components. Thus, instead of a single warning sound being emitted only indicating a general malfunction, the sounds emitted are capable of directing the user to the problem component. The sounds may therefore include different generalized sounds or may comprise a series of words dictating which component has triggered the alert.

Within the system 10, the detectors 68 of the monitoring and warning system may include a large number sensors having a variety of functions. For example, within the milking assembly 14, a vacuum sensor 74 fluidly coupled between the vacuum pump 20 and the reserve tank 22 measures vacuum pressure being exerted by the vacuum pump 20 and will inform a user whether or not the vacuum pump 20 is functioning at correct capacities. Should the negative pressure not be strong enough, the pulsator 25 and milking unit 26 will not function properly to draw milk while too strong of pressure could be harmful to livestock as well as comprise the ability of the milk to remain in the receiver jar 32. A milk temperature sensor 76 is in thermal communication with the milk to determine a temperature of the milk when the milk is in the milk tank 36. The milk temperature sensor may be positioned either within the milk tank 36 or before the milk tank 36 should the plate cooler 46 be used to chill the milk before it enters the milk tank 36. Regardless, the milk temperature sensor 76 will be used to ensure that the milk is of the proper, cooled temperature while being held in the milk tank 36 to prevent bacterial growth. It should be noted that the term “temperature sensor” used herein may include any conventional means for measuring temperatures including, without limitation, negative temperature coefficient thermistors, resistance temperature detectors, thermocouples, semiconductor-based sensors, and the like.

A milk line temperature sensor 78 may be utilized in addition to the milk temperature sensor 76 and be in fluid communication with the milk line 30, typically after the milk has been cooled. Additionally, of great importance, is a vacuum amperage sensor 80 is electrically coupled to the vacuum pump 20. The term “amperage sensor” or variations thereof herein may include conventional current transformers or transducers, for example. Incorrect vacuum amperage would alert the user that the vacuum pump 20 is either functioning improperly or is in imminent danger of doing so. Should the vacuum pump 20 fail, the entire milking procedure would stall due to the milking units 26 becoming non-functional as well as their being no suction for moving milk through the milk line.

Additional sensors may include at least one coolant temperature sensor 82 which is in thermal connection with the refrigerant. This may include multiple coolant temperature sensors 82, 83 as the refrigerant is monitored both before and after the compressor 44 to ensure its temperature is maintained through the cooling assembly 16. A low pressure coolant transducer 84 is in fluid communication with the cooling assembly 16 to determine a refrigerant level of the cooling assembly 16 and is positioned in line between the compressor 44 and the evaporator 40. A high pressure coolant transducer 86 is in fluid communication with the cooling assembly 16 to determine the refrigerant level of the cooling assembly 16 and is positioned in line between the compressor 44 and the condenser 42. The low 84 and high 86 pressure coolant transducers will measure the pressure within the coolant lines to ensure enough coolant is traveling through the evaporator 40 and condenser 42. Though pressure transducers are named herein, they may be replaced with other common pressure sensors such as pressure transmitters, pressure senders, pressure indicators, piezometers and manometers. Additionally, a compressor amperage sensor 88 is electrically coupled to the compressor 44 to again ensure proper functioning of the cooling assembly's compressor 44. Each of the above ensure that the coolant is properly flowing and being cooled to thereafter chill the milk for storage. Typically, coolant pumps 90 are utilized to move coolant from and between a coolant holding tank 54 and the evaporator 40 and plate cooler 46. The coolant pumps 90 include amperage sensors 92 for measuring the amperage of the coolant pumps 90 during their operation. A coolant storage temperature sensor 94 is mounted on the coolant tank 54 for measuring the temperature of the coolant therein.

As previously indicated, the secondary cooling assembly 17 would also include coolant temperature sensors 82 and 83, a low pressure coolant transducer 84, a high pressure coolant transducer 86, and a compressor amperage sensor 88.

For the sanitizing assembly 18, a plurality of volume sensors 96 is used for the hold tanks 60 to ensure that hold tanks 60 include the required amount of cleaning agents. Each of the hold tanks 60 has an interior space for storing one of the cleaning agents and the volume sensors 96 are communication with the interior spaces measure the amount of cleaning agent in each hold tank 60. The volume sensors 96 may include conventional floats or sonar sensors which detect the surface of the cleaning agents. A plurality of pump amperage sensors 98 may be provided and each of the cleaning pumps 62 is electrically coupled to one of the pump amperage sensors 98. Thus, should any of the cleaning pumps fail or begin to mechanically falter, the pump amperage sensors 98 will detect the same and warn the user that the sanitizing assembly 18 may not be functioning properly.

Though not typically considered warning sensors, a milk flow meter 100 as well as a tank level sensor 100 may be in fluid communication with the milk line 30 and milk tank 36, respectively. The milk flow meter 100 may be used to indicate the amount of fluid per unit of time is flowing through the milk line 30 as well as monitor a total amount of milk being delivered to the milk tank 36 over a predetermined amount of time. The tank level sensor 100 will alert users should the milk tank 36 become filled to near capacity. The tank level sensor 100 may include a float, IR sensor, or other measuring devices conventionally used for measuring the amount of fluid within a tank. The tank level sensor 102 and the milk flow meter 100 are each in communication with the processor 66.

In use, the milk collection apparatus 12 is used in a conventional manner to milk a plurality of cows. However, the system 10 allows a user to be immediately notified should a failure within the milking 14, cooling 16 or sanitizing 18 assemblies occur. Furthermore, the user will receive an alert specifically providing details as to where the failure or concern, i.e. a measurement outside of the prescribed parameters, is occurring such that the user is more quickly directed to the failing component. The alert may be received remote from the milk collection apparatus 12 and the problems diagnosed remotely to allow instructions to be sent to someone on-site to correct the malfunction.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A milking equipment assembly monitoring system configured to monitor multiple components of the milking equipment assembly independently of each other, the system including:
a milk collection apparatus for milking cattle, the milk collection apparatus including a plurality of components in fluid communication with each other for milking a cow and to transport milk for storage;
a monitoring and warning system being in communication with the milk collection apparatus to detect proper functioning of the components of the milk collection apparatus, the monitoring and warning system indicating operation of multiple ones of the components independently of other ones of the components;
the monitoring and warning system including a processor and a plurality of detectors, wherein at least some of the components include a detector capable of reading a measurement being characteristic of that component, the detectors sending the measurements of the components to the processor, the processor being programmed to determine if any of the measurements are outside of a standard parameter; and
an interface being in communication with the processor providing an alert when one of the measurements is outside of a standard parameter for that measurement, the alert being an audible and/or visual alert, the alert indicating the component associated with the measurement outside of the standard parameter.

2. The milking equipment assembly monitoring system according to claim 1, wherein:
the plurality of components includes:
a vacuum pump fluidly coupled to a reserve tank;
a pulsator line fluidly coupled to the reserve tank;
a milking unit fluidly coupled to the pulsator line;
a milk line fluidly coupled to the milking unit and to the reserve tank;
a receiver jar fluidly coupled to the milk line between the milking unit and the reserve tank;
a milk pump fluidly coupled to the receiver jar;
a milk tank fluidly coupled to the milk pump, wherein milk received by the milking unit flows into the receiver jar from the milk line and the milk pump pumps the milk from the receiver jar into the milk tank;
a cooling assembly being in thermal communication with the milk, the cooling assembly including an evaporator, a condenser, and a compressor in fluid communication with each other;
the detectors of the monitoring and warning system including:
a vacuum sensor being fluidly coupled to the vacuum between the vacuum pump and the reserve tank to measure vacuum pressure; and
a milk temperature sensor being in thermal communication with the milk to determine a temperature of the milk when the milk is in the milk tank.

3. The milking equipment assembly monitoring system according to claim 2, wherein the detectors of the monitoring and warning system further include a vacuum amperage sensor being electrically coupled to the vacuum.

4. The milking equipment assembly monitoring system according to claim 2, wherein the detectors of the monitoring and warning system further include at least one coolant temperature sensor being in thermal connection with the refrigerant.

5. The milking equipment assembly monitoring system according to claim 2, wherein the detectors of the monitoring and warning system further include a low pressure coolant transducer being in fluid communication with the cooling assembly to determine a refrigerant level of the cooling assembly and being positioned in line between the compressor and the evaporator.

6. The milking equipment assembly monitoring system according to claim 2, wherein the detectors of the monitoring and warning system further include a high pressure coolant transducer being in fluid communication to determine the refrigerant level of the cooling assembly and being positioned in line between the compressor and the condenser.

7. The milking equipment assembly monitoring system according to claim 2, wherein the detectors of the monitoring and warning system further include a compressor amperage sensor being electrically coupled to the compressor.

8. The milking equipment assembly monitoring system according to claim 2, wherein:
the milk collection apparatus further includes:
a sanitizing assembly being in fluid communication with the milk line, the sanitizing assembly including:
a plurality of hold tanks each configured to contain one of a plurality of cleaning agents;
a plurality of cleaning pumps, each of the cleaning pumps being fluidly coupled to at least one of the hold tanks, each of the cleaning pumps being fluidly coupled to the milk line for pumping cleaning agents into the milk line;
wherein the detectors of the monitoring and warning system further include a plurality of volume sensors, each of the hold tanks having an interior space for storing one of the cleaning agents being in communication with one of the volume sensors to measure the amount of cleaning agent in each hold tank.

9. The milking equipment assembly monitoring system according to claim 8, wherein the detectors of the monitoring and warning system further include a plurality of pump amperage sensors, each of the cleaning pumps being electrically coupled to one of the pump amperage sensors.

10. The milking equipment assembly monitoring system according to claim 1, wherein the interface further includes a display being electrically coupled to the processor to display the measurements.

11. The milking equipment assembly monitoring system according to claim 1, wherein the interface further includes a sound emitter being in communication with the processor, the sound emitter emitting one of a plurality of sounds when a measurement is outside of the standard parameter for that measurement, each of the sounds being different with respect to each other and being associated with only one of the components.

12. The milking equipment assembly monitoring system according to claim 1, wherein the interface further includes a wireless transmitter being electrically coupled to the processor to wirelessly transmit the measurements to a receiver for remote viewing of the measurements.

13. The milking equipment assembly monitoring system according to claim 12, wherein:
the plurality of components includes:
a vacuum pump fluidly coupled to a reserve tank;
a pulsator line fluidly coupled to the reserve tank;
a milking unit fluidly coupled to the pulsator line;
a milk line fluidly coupled to the milking unit and to the reserve tank;
a receiver jar fluidly coupled to the milk line between the milking unit and the reserve tank;
a milk pump fluidly coupled to the receiver jar;
a milk tank fluidly coupled to the milk pump, wherein milk received by the milking unit flows into the receiver jar from the milk line and the milk pump pumps the milk from the receiver jar into the milk tank;
a cooling assembly being in thermal communication with the milk, the cooling assembly including an evaporator, a condenser, and a compressor in fluid communication with each other;
the detectors of the monitoring and warning system including:
a vacuum sensor being fluidly coupled to the vacuum between the vacuum pump and the reserve tank to measure vacuum pressure; and
a milk temperature sensor being in thermal communication with the milk to determine a temperature of the milk when the milk is in the milk tank.

14. The milking equipment assembly monitoring system according to claim 13, wherein:
the milk collection apparatus further includes:
a sanitizing assembly being in fluid communication with the milk line, the sanitizing assembly including:
a plurality of hold tanks each configured to contain one of a plurality of cleaning agents;
a plurality of cleaning pumps, each of the cleaning pumps being fluidly coupled to at least one of the hold tanks, each of the cleaning pumps being fluidly coupled to the milk line for pumping cleaning agents into the milk line;
wherein the detectors of the monitoring and warning system further include a plurality of volume sensors, each of the hold tanks having an interior space for storing one of the cleaning agents being in communication with one of the volume sensors to measure the amount of cleaning agent in each hold tank.

15. A milking equipment assembly monitoring system configured to monitor multiple components of the milking equipment assembly independently of each other, the system including:
a milk collection apparatus for milking cattle, the milk collection apparatus including a plurality of components in fluid communication with each other for milking a cow and to transport milk for storage, the plurality of components including:
a vacuum pump fluidly coupled to a reserve tank;
a pulsator line fluidly coupled to the reserve tank;
a milking unit fluidly coupled to the pulsator line;
a milk line fluidly coupled to the milking unit and to the reserve tank;
a receiver jar fluidly coupled to the milk line between the milking unit and the reserve tank;
a milk pump fluidly coupled to the receiver jar;
a milk tank fluidly coupled to the milk pump, wherein milk received by the milking unit flows into the receiver jar from the milk line and the milk pump pumps the milk from the receiver jar into the milk tank;
a cooling assembly being in thermal communication with the milk, the cooling assembly including an evaporator, a condenser, and a compressor in fluid communication with each other;
a sanitary milk trap being in fluid communication with the milk line between the receiver jar and the reserve tank;
a sanitizing assembly being in fluid communication with the milk line, the sanitizing assembly including:
a plurality of hold tanks each configured to contain one of a plurality of cleaning agents;
a plurality of cleaning pumps, each of the cleaning pumps being fluidly coupled to at least one of the hold tanks, each of the cleaning pumps being fluidly coupled to the milk line for pumping cleaning agents into the milk line;
a monitoring and warning system being in communication with the milk collection apparatus to detect proper functioning of the components of the milk collection apparatus, the monitoring and warning system indicating operation of each component independently of other ones of the components;
the monitoring and warning system including a processor and a plurality of detectors, wherein at least some of the components include a detector capable of reading a measurement being characteristic of that component, the detectors sending the measurements of the components to the processor, the processor being programmed to determine if any of the measurements are outside of a standard parameter;
a display being electrically coupled to the processor to display the measurements;
a sound emitter being in communication with the processor, the sound emitter emitting one of a plurality of sounds when a measurement is outside of the standard parameter for that measurement, each of the sounds being different with respect to each other and being associated with only one of the components;
a wireless transmitter being electrically coupled to the processor to wirelessly transmit the measurements to a receiver for remote viewing of the measurements;
the detectors of the monitoring and warning system including:
a vacuum sensor being fluidly coupled to the vacuum between the vacuum pump and the reserve tank to measure vacuum pressure;
a milk temperature sensor being in thermal communication with the milk to determine a temperature of the milk when the milk is in the milk tank;
a vacuum amperage sensor being electrically coupled to the vacuum;
at least one coolant temperature sensor being in thermal connection with the refrigerant;
a low pressure coolant transducer being in fluid communication with the cooling assembly to determine a refrigerant level of the cooling assembly and being positioned in line between the compressor and the evaporator;
a high pressure coolant transducer being in fluid communication to determine the refrigerant level of the cooling assembly and being positioned in line between the compressor and the condenser;
a compressor amperage sensor being electrically coupled to the compressor;
a plurality of volume sensors, each of the hold tanks having an interior space for storing one of the cleaning agents being in communication with one of the volume sensors to measure the amount of cleaning agent in each hold tank; and
a plurality of pump amperage sensors, each of the cleaning pumps being electrically coupled to one of the pump amperage sensors.
